(19) United States
(12) Patent Application Publication
(10) Pub. No.: US 2008/0220473 A1
(43) Pub. Date: Sep. 11, 2008

(54) Endo-N-Acetyl-Beta-D-Glucosaminidase Enzymes of Filamentous Fungi

(75) Inventors: Marc Claeyssens, Gent (BE); Ingeborg Stals, Bellegem (BE)

Correspondence Address: CLARK & ELBING LLP, 101 FEDERAL STREET, BOSTON, MA, 02110, US

(73) Assignee: UNIVERSITEIT GENT, Gent (BE)

(21) Appl. No.: 11/719,083

(22) PCT Filed: Nov. 9, 2005
(86) PCT No.: PCT/BE05/00160

§ 371 Date: May 10, 2007

Related U.S. Application Data
(60) Provisional application No. 60/626,752, filed on Nov. 10, 2004.
(60) Provisional application No. 60/682,963, filed on May 20, 2005.

(51) Int. Cl. C12P 21/00; C07H 21/00; C12N 9/14; C12N 9/42; C07K 16/00; C12N 1/19; C12P 1/04; C12P 1/02

(52) U.S. Cl. 435/69.1; 536/23.74; 435/262; 435/195; 435/209; 530/387.9; 435/254.2; 435/170; 435/171

(57) ABSTRACT

The present invention discloses mannosyl-glycoprotein endo-beta-N-acetylglucosamidase (E.C.3.2.1.96, endo-N-acetyl-beta-D-glucosaminidase acting on the di-N-acetylchitobiosyl part of N-linked glycans) from filamentous fungi such as Trichoderma reesei.

### FIELD OF THE INVENTION

The present invention relates to N-deglycosylating enzymes from filamentous fungi and fragments thereof for use in industrial applications. The present invention provides nucleotides encoding such enzymes of the invention, as well as methods involving the use of the enzymes of the invention.

### BACKGROUND

Saprophytic micro-organisms produce and secrete a variety of hydrolytic enzymes to degrade organic substrates. Organisms producing cellulases and hemicellulases are of particular interest because of their industrial potential and use in degradation of biomass for e.g. bio-fuel production. Among the most prolific producers of biomass-degrading enzymes is the filamentous fungus Trichoderma reesei (now called Hypocrea jecorina). The cellulases produced act synergistically with beta-glucosidases to break down cellulose to glucose providing nutrients for growth and contributing to carbon recycling in nature.

All T. reesei cellulases but one, are glycoproteins with a typical bi-modular structure: a flexible linker peptide connects the catalytic module (core) with a carbohydrate binding module (CBM). Whereas N-glycosylation seems to be restricted to Asn consensus sequences present in the core domain, O-glycosylation is predominantly present in the Ser and Thr-rich linker region. The CBM is generally not glycosylated. Due to heterogeneity in N- and O-glycan structures, cellulases occur as glycosylated variants. The occurrence of phosphate, sulfate and phosphodiester residues can result in different iso-(fosfo)forms of one enzyme.

It has been shown that the glycosylation of Cel7A (cellobiohydrolase I) from Trichoderma reesei varies considerably when the fungus is grown under different conditions (Stals et al., (2004a) Glycobiology 14, 713-737). Fully N- and O-glycosylated Cel7A could only be isolated from minimal medium and probably reflects the initial complexity of the protein upon leaving the glycosynthetic pathway (Stals et al., (2004b) Glycobiology 14, 725-724). An array of hydrolytic activities, present in the extra-cellular media is responsible for post-secretorial modifications in other cultivation conditions: alpha-(1→2)-mannosidase, alpha-(1→3)-glucosidase and an endo H-type activity participate in N-deglycosylation (core), while a phosphatase and a mannosidase are probably responsible for hydrolysis of O-glycans (linker) (Stals et al., (2004a), above. The effects are most prominent in corn steep liquor enriched media, wherein the pH is close to the pH optimum (5-6) of these extracellular hydrolases.

The presence of a mannosyl glycoprotein endo-N-acetylglucosaminidase type activity (EC 3.2.1.96) in the extracellular medium of T. reesei had been suggested in Klarskov et al. (1997, Carbohydr. Res. 752, 349-368) and Harrison et al., (1997, Eur. J. Biochem. 256, 119-127) as an explanation for the presence of single N-acetylglucosamine residues. Recently, it was demonstrated that only in growth media with a pH value near 5, this activity was indeed responsible for the intensive deglycosylation observed (Stals et al., (2004a), above) Partially occupied glycosylation sites contribute further to the microheterogeneity of cellulases evidencing the existence of different glycoforms of one enzyme (Hui et al., (2001) J. Chrom. B 752, 349-368).

To elucidate the structure and function of the oligosaccharide moieties of glycoproteins, exoglycosidases and endoglycosidases are generally used. The enzymes acting on the di-N-acetylchitobiosyl part of N-linked glycans appear to be the most useful in determining the relation between structure and function of glycoproteins. These enzymes, endo-N-acetyl-beta-D-glucosaminidase and peptide-N-(N-acetyl-beta-D-glucosaminyl) asparagine amidase are qualified as the restriction enzymes of the carbohydrate world. Although they have proven be useful tools for studying glycoproteins, little attention has been given to the understanding of their possible roles in the physiology of the cells producing them. E.g. the widespread occurrence of the sugar coat in hydrolytic enzymes from fungi implies that they fulfil an essential function. Contribution to stability, generation of a rigid linker conformation and protection from proteolytic attack have been reported as essential functions of O-glycosylation of the linker region. The importance of N-glycosylation for secretion or stability is less clear. However, many fungi seem to possess an endo-N-acetyl-beta-D-glucosaminidase involved in the N-glycan degradation pathway. So the potential substrates for the endo-N-acetyl-beta-D-glucosaminidase activity are widespread.

Bacteria and fungi release in their environment hydrolytic enzymes which decay plant and animal tissues and ensure the removal of protective oligosaccharide moieties thereby allowing the bacteria and fungi to sequester small peptides and amino acids from exogenous protein to satisfy energy and nitrogen requirements.

The endo-N-acetyl-beta-D-glucosaminidase present in the medium of T. reseei could thus contribute to the accessibility of the peptide part of N-glycosylproteins; Another possibility is that by releasing discrete oligosaccharides from native N-glycosylproteins excreted by the fungus, endoglycosidases contribute to the generation of a family of distinct signals.

### SUMMARY OF THE INVENTION

The present invention relates to endo-beta-N-acetylglucosamidase enzymes and their use in industry.

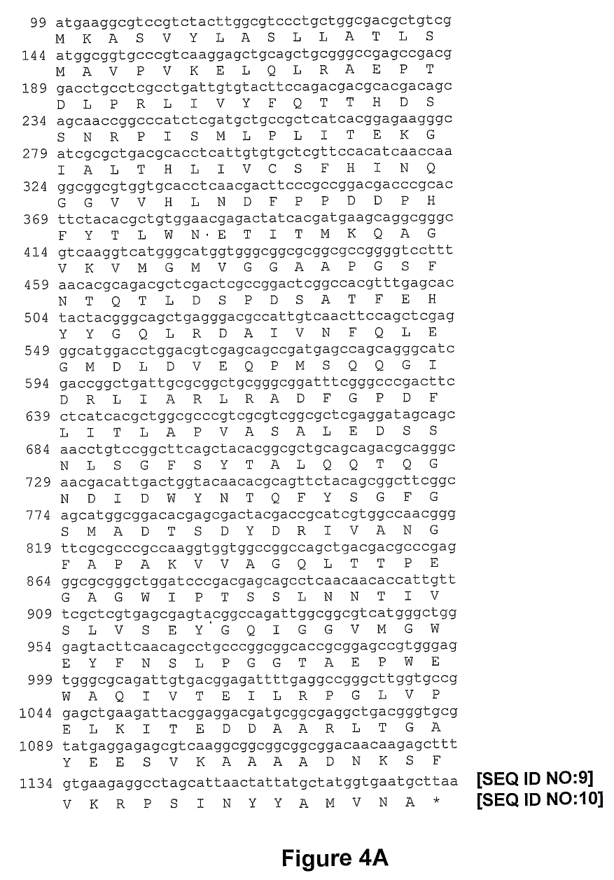

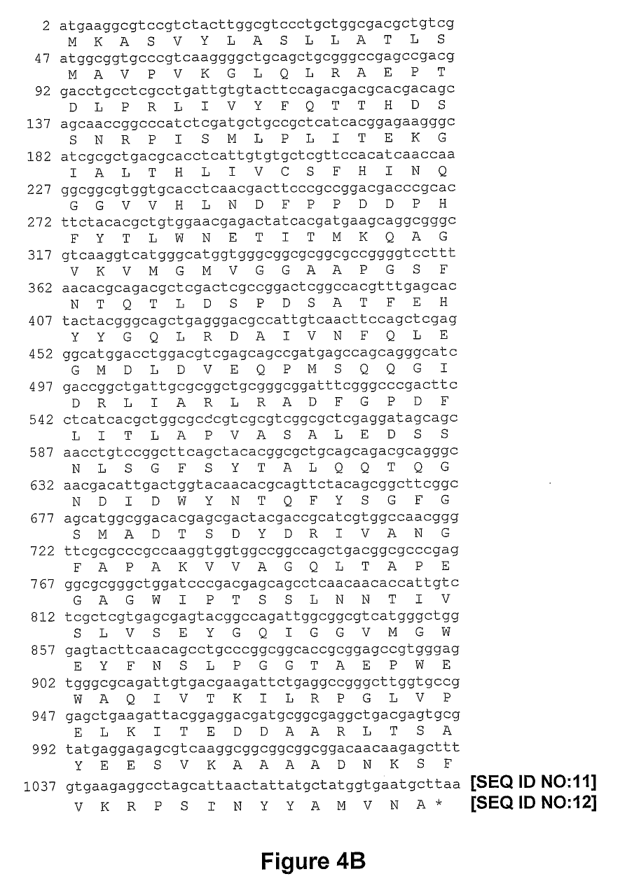

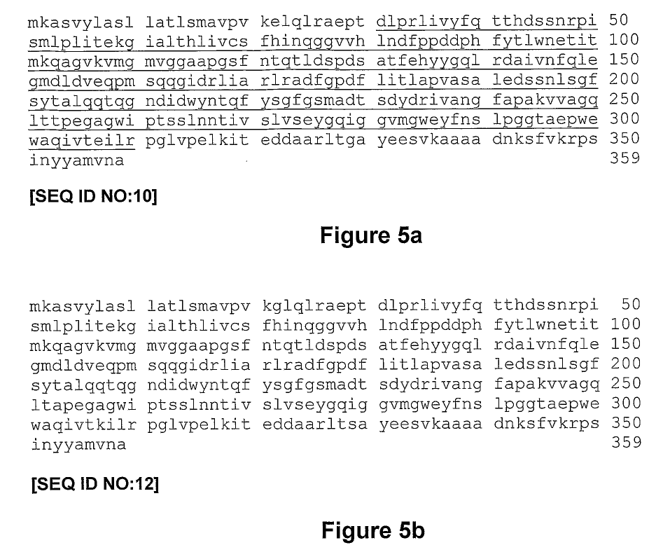

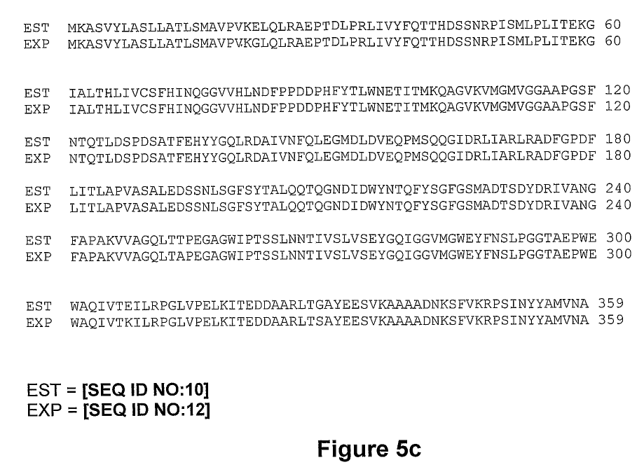

A first aspect of the invention provides isolated polypeptides of filamentous fungi, more particularly of Trichoderma reesei, having mannosyl-glycoprotein endo-beta-N-acetylglucosamidase activity. Specific embodiments of the invention relate to proteins having an amino acid sequence as depicted in FIG. 4A [SEQ ID NO:10] or 4B [SEQ ID NO:12] or an amino acid sequence with at least 70% sequence similarity to the amino acid sequence depicted in FIG. 4A or 5A [SEQ ID NO:10] or 4B or 5B [SEQ ID NO:12] or a fragment thereof with mannosyl-glycoprotein endo-beta-N-acetylglucosamidase activity. Further specific embodiments relate to polypeptides having mannosyl-glycoprotein endo-beta-N-acetylglucosamidase activity and having an amino acid sequence corresponding to a sequence as depicted in FIG. 4A or 5A [SEQ ID NO:10] or 4B or 5B [SEQ ID NO:12] which has been N-terminally and/or C-terminally truncated. Accordingly, the present invention also provides specific antibodies, directed against the protein and polypeptide sequences of the invention.

A second aspect of the invention provides isolated nucleotide sequences encoding the enzymes of the invention. More particularly the invention provides isolated polynucleotides encoding a protein of a filamentous fungus, the encoded protein having an amino acid sequence as depicted in FIG. 5A [SEQ ID NO:10] or 5B [SEQ ID NO:12], or an amino acid sequence having at least 70% sequence similarity therewith. Further embodiments relate to nucleotide sequences encoding a fragment of the aforementioned protein, which protein fragment has mannosyl-glycoprotein endo-beta-N-acetylglucosamidase activity. Particular embodiments of the invention relate to the isolated polynucleotides comprising the nucleotide sequences depicted in FIG. 4A [SEQ ID NO:9] or 4B [SEQ ID NO:11] or a sequence with at least 70% sequence identity therewith. Most particular embodiments relate to polynucleotide sequences isolated from Trichoderma sp. encoding a protein having mannosyl-glycoprotein endo-beta-N-acetylglucosamidase activity.

Yet another aspect of the invention relates to the use of the nucleotide sequences encoding the endo-beta-N-acetylglucosamidase activity in the recombinant production of the enzyme. According to a particular embodiment the nucleotide sequences are introduced into a suitable host under control of a promoter which ensures expression, more particularly overexpression of the enzyme in said host. The recombinantly produced enzyme can then be purified from the host.

Yet another aspect of the invention relates to the use of the protein or polypeptide sequences described above in the degradation of organic material. Specific embodiments of the degradation of organic material using the enzymes of the invention include degradation processes performed in a medium with a pH between 4.5 and 5.5.

A particular embodiment of the present invention relates to the use of the protein or polypeptide sequence having endo-beta-N-acetylglucosamidase activity in the production of bio-fuel as well as to the biofuel made by the process. Thus, the present invention provides methods for the production of bio-fuel, which encompass the step of degrading organic material with a polypeptide according to the invention. Additionally, the invention provides a process for the production of bio-fuel which comprises the step of introducing into a micro-organism a sequence encoding a protein having endo-beta-N-acetylglucosamidase activity, said protein having a sequence with at least 80% sequence identity to the amino acid sequence depicted in FIG. 5A [SEQ ID NO:10] or 5B [SEQ ID NO:12] or ensuring over-expression of said protein in said micro-organism. According to specific embodiments such organism is a yeast or bacterial cell. Optionally, other sequences can be introduced into said micro-organism which Thus, the present invention provides biofuel made by the processes of the invention, more particularly made by degradation of organic material by use of the protein having endo-beta-N-acetylglucosamidase activity.

Yet another aspect of the invention relates to the generation of an endo-beta-N-acetylglucosamidase deletion strain of a filamentous fungus for the production of an enzyme with an enhanced glycosylation and/or increased stability. Specific embodiments of this aspect of the invention relate to the production of cellulases with enhanced glycosylation and/or increased stability. More specifically the filamentous fungus is T. reesei.

Yet another aspect of the invention relates to expression systems, more particularly transgenic cells, such as bacteria or yeast cells, which comprise either a foreign DNA comprising the nucleotide sequence encoding a protein having endo-beta-N-acetylglucosamidase activity of the invention or in which an endogenous sequence encoding a protein having endo-beta-N-acetylglucosamidase activity is placed under control of a foreign promoter.

### DETAILED DESCRIPTION OF THE INVENTION

Figure Legends:

The following Figures illustrate the invention but are not to be interpreted as a limitation of the invention to the specific embodiments described therein.

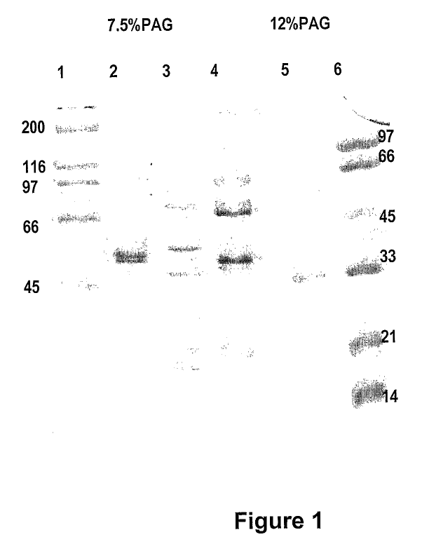

FIG. 1: purification of T reesei Endo T on SDS-polyacrylamide gel under reducing conditions according to an embodiment of the invention. Lane 1: standard proteins; lane 2: crude medium; lane 3: non-bound fraction on avicel; lane 4: fractions pooled after DEAE-sepharose FF chromatography; Lane 5: purified Endo T after chromatography on the Biogel P-100 column; lane 6: low molecular weight standard proteins. The gel was stained with Coomassie blue.

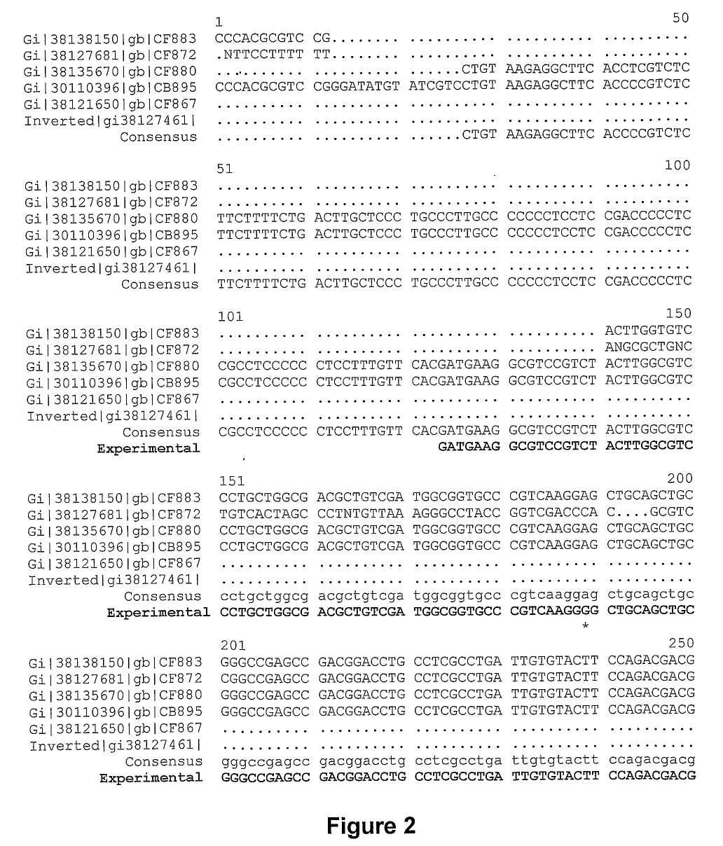

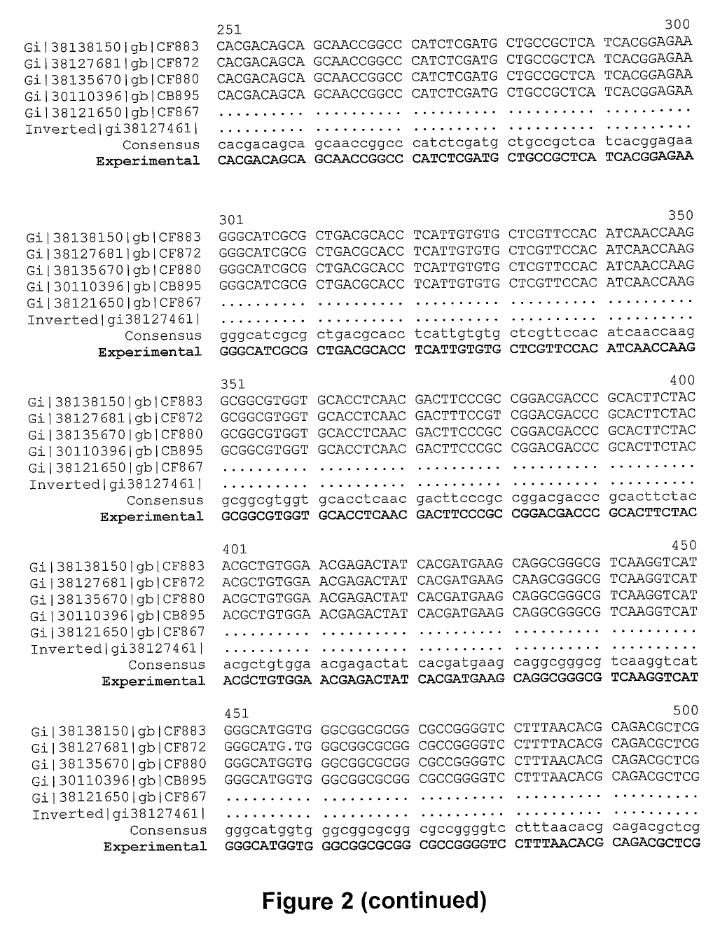

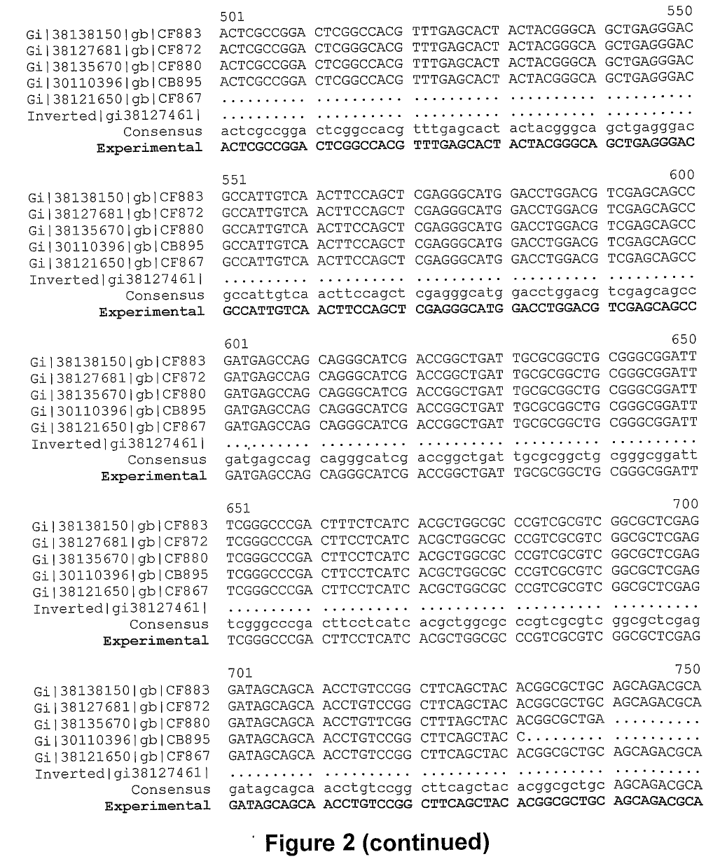

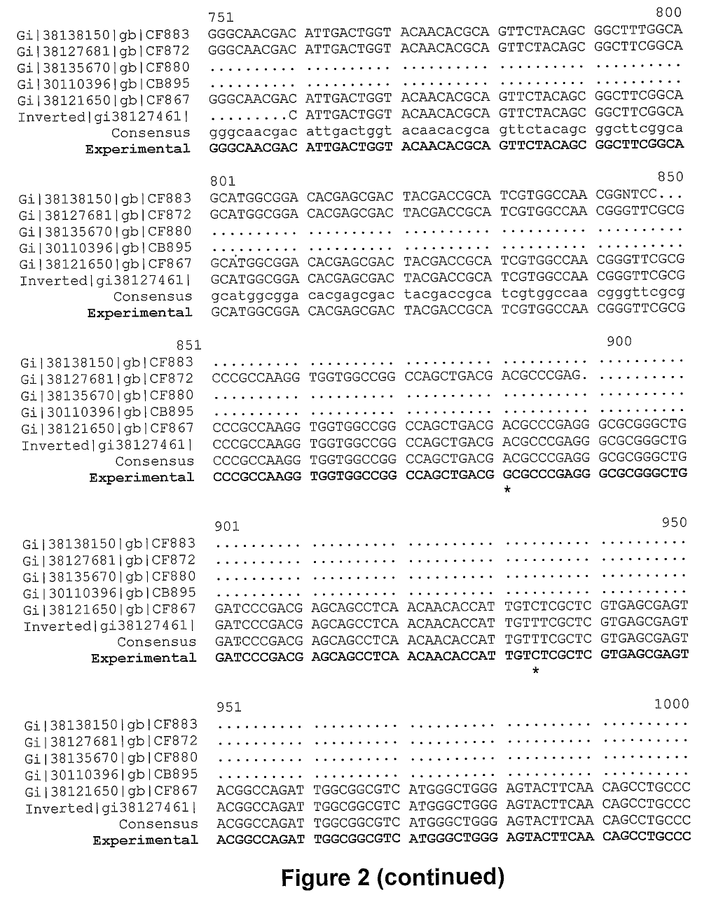

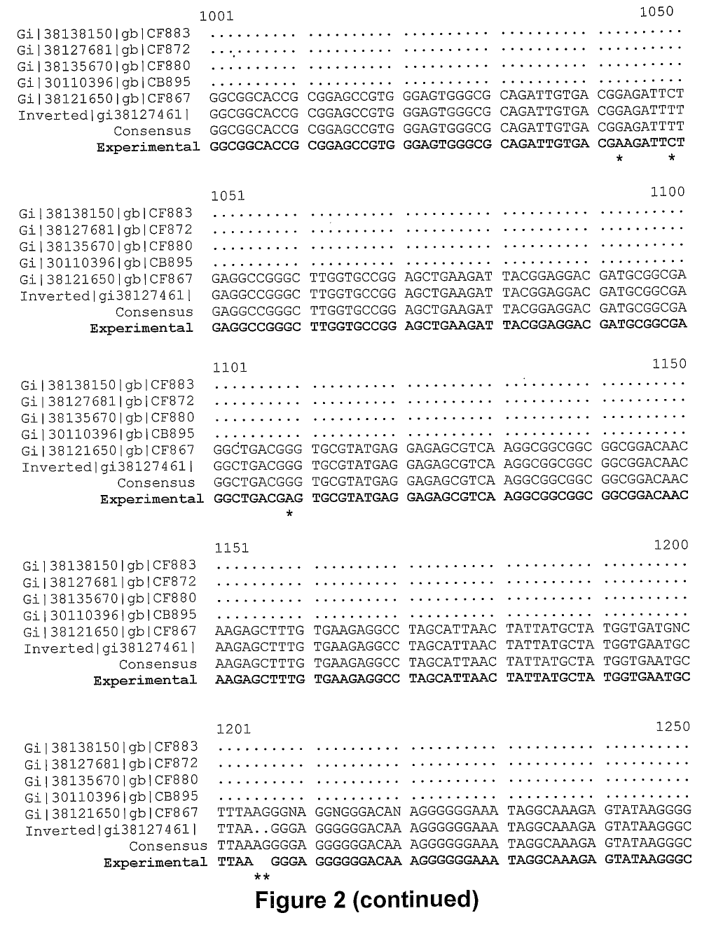

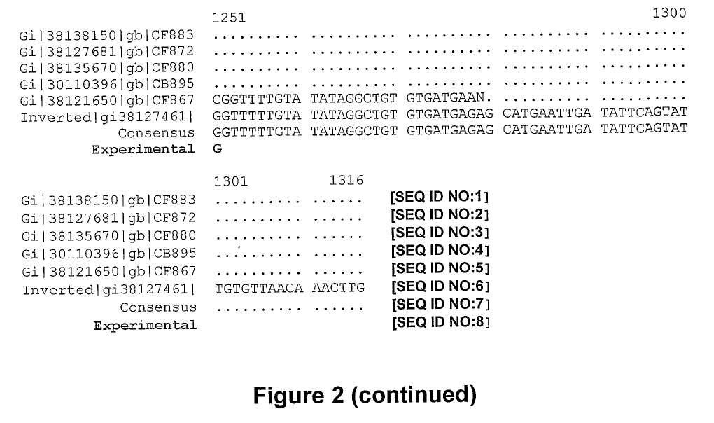

FIG. 2: alignment of EST cDNA clones [SEQ ID NO:1 to 6] coding for peptide sequences of EndoH (determined by Mass spectrometry) according to an embodiment of the invention. A consensus sequence encoding a theoretical coding sequence is indicated with “consensus” [SEQ ID NO:7]. The sequence obtained via molecular biology techniques is indicated with “experimental” [SEQ ID NO:8].

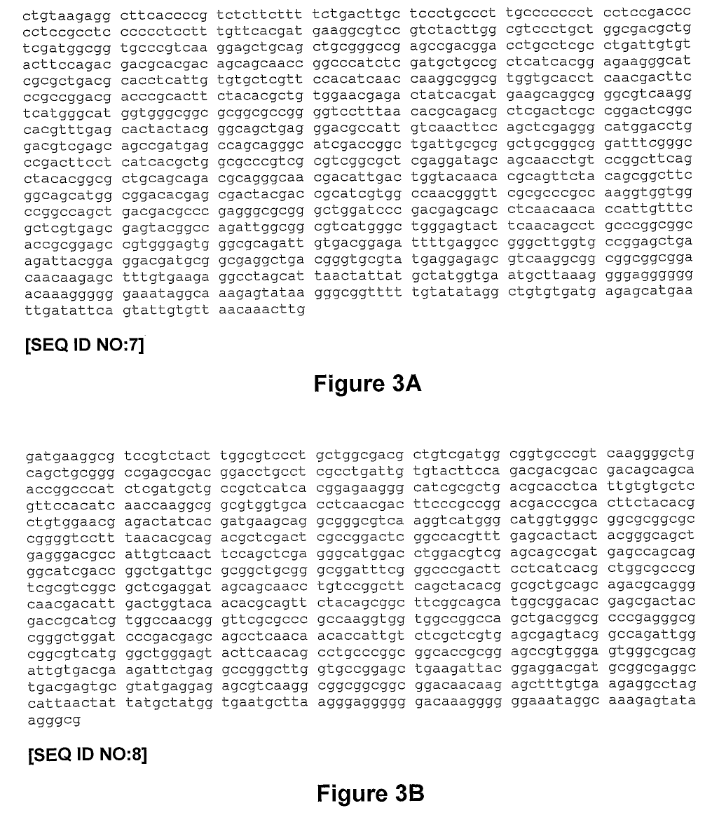

FIG. 3: A. ‘consensus’ sequence [SEQ ID NO:7] derived from the alignment in FIG. 2. according to an embodiment of the invention, B. cDNA sequence of T. reesei Endo T [SEQ ID NO:8] as obtained via recombinant molecular biology techniques according to an embodiment of the invention (‘experimental’).

FIG. 4: A. Open reading frame in the cDNA sequence of T. reesei Endo T [SEQ ID NO:9], assembled from EST clones as shown in FIG. 2, and the corresponding amino acid sequence [SEQ ID NO:10], according to an embodiment of the invention; B. open reading frame in the cDNA sequence of the cloned gene of T. reesei Endo T [SEQ ID NO:11], shown in FIG. 2 and the corresponding amino acid sequence [SEQ ID NO:12], according to an embodiment of the invention.

FIG. 5: (a) putative T. reesei Endo T sequence [SEQ ID NO:10], according to an embodiment of the invention; location of the putative glycoside hydrolase family 18 domain sequence underlined); (b) amino acid sequence of T. reesei Endo T [SEQ ID NO:12] encoded by the experimental DNA sequence, according to an embodiment of the invention; (c) Sequence alignment between the translated protein sequence (EST) of the EST assembled cDNA sequence and the translated protein (exp) sequence of experimental sequence [SEQ ID NO:10 versus SEQ ID NO:12]. Differences between the sequences are indicated with *.

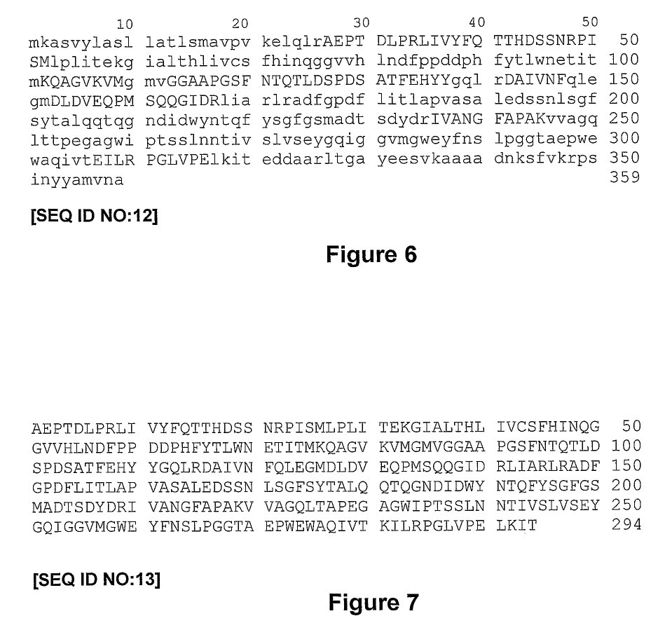

FIG. 6: location of the experimentally determined peptide sequences in the amino acid sequence of T. reesei Endo T, according to an embodiment of the invention (sequence confirmed by Mass spectrometry between residue 27 and 316 (capitals))

FIG. 7: amino acid sequence of mature T. reesei Endo T [SEQ ID NO:13] based on aminoterminal sequence determination and Mr determined by Mass spectrometry, according to an embodiment of the invention.

### DEFINITIONS

“Endo T” of T. reesei as used herein refers to, an enzyme with the activity of Mannosyl-glycoprotein endo-beta-N-acetylglucosamidase. (E.C.3.2.1.96) obtainable from Trichoderma reesei. This reaction is the endohydrolysis of the di-N-acetylchitobiosyl unit in high-mannose glycopeptides and glycoproteins containing the -[Man(GlcNAc)₂]Asn- structure. One N-acetyl-D-glucosamine residue remains attached to the protein; the rest of the oligosaccharide is released intact. The enzymatic activity is also referred to as endo-beta-N-acetylglucosaminidase or di-N-acetylchitobiosyl beta-N-acetylglucosaminidase activity.

This activity belongs to EC.3.2.1.96 with members in the glycoside hydrolase families 18, 73 and 85 (see Table 1 below).

TABLE 1
Glycosidase hydrolase families

| | Glycoside | | Glycoside |
|---|---|---|---|
| CAZy | Hydrolase Family | Glycoside Hydrolase | Hydrolase Family |
| Family | 18 | Family 73 | 85 |
| Known | chitinase (EC | endo-β-N- | endo-β-N-acetyl- |
| Activities | 3.2.1.14); | acetylglucosaminidase | glucosaminidase |
| | endo-β-N-acetyl- | (EC 3.2.1.96); β-1,4-N- | (EC 3.2.1.96) |
| | glucosaminidase | acetylmuramoylhydrolase | |
| | (EC 3.2.1.96); | (EC 3.2.1.17). | |
| | non-catalytic | | |
| | proteins: xylanase | | |
| | inhibitors; | | |
| | concanavalin B; | | |
| | narbonin | | |
| Mechanism | Retaining | Not known | probably retaining |
| Catalytic | Carbonyl oxygen | | Not known |
| Nucleophile/ | of C-2 acetamido | | |
| Base | group of substrate | | |
| Catalytic | Glu (experimental) | Not known | Not known |
| Proton Donor | | | |
| 3D | Available (see | Not known | Not known |
| Structure Status | PDB). Fold (β/α)₈ | | |
| Clan | GH-K | Not available | Not available |
| Statistics | CAZy(944); | CAZy(221); | CAZy(24); |
| | GenBank/GenPept | GenBank/GenPept | GenBank/GenPept |
| | (1492); Swissprot | (390); Swissprot (84) | (49); Swissprot |
| | (708); PDB (86); 3D(22) | | (20) |

The “sequence identity” of two sequences as used herein relates to the number of positions with identical nucleotides or amino acids divided by the number of nucleotides or amino acids in the shorter of the sequences, when the two sequences are aligned. The alignment of two nucleotide sequences is performed by the algorithm as described by Wilbur and Lipmann (1983) Proc. Natl. Acad. Sci. U.S.A. 80:726, using a window size of 20 nucleotides, a word length of 4 nucleotides, and a gap penalty of 4.

Two amino acids are considered as “similar” if they belong to one of the following groups GASTCP; VILM; YWF; DEQN; KHR. Thus, sequences having “sequence similarity” means that when the two protein sequences are aligned the number of positions with identical or similar nucleotides or amino acids divided by the number of nucleotides or amino acids in the shorter of the sequences, is higher than 80%, preferably at least 90%, even more preferably at least 95% and most preferably at least 99%, more specifically is 100%.

A “foreign” DNA sequence as used herein refers to the fact that it has been introduced into the DNA of the cell e.g. by molecular biology techniques and/or by recombination. A foreign promoter when referring to the nucleotide sequence encoding a protein or polypeptide is a promoter that is not naturally associated with that coding sequence in a cell.

The present invention discloses the purification and the isolation of an endo-beta-N-acetylglucosamidase enzyme from Trichoderma reesei. This enzyme, named Endo T, exhibits strong endohydrolytic activity on oligomannosidic-type glycoproteins but does not hydrolyze hybrid- and complex-type glyco-asparagines. The invention also discloses the characterization of the protein at the amino acid level as well as the characterization at the DNA level, by in silico assembly as well as by molecular biology techniques.

In a first aspect, the present invention thus provides proteins and protein fragments with endo-beta-N-acetylglucosamidase activity which have an amino acid sequence which is at least 60%, particularly at least 70%, most particularly at least 80%, especially at least 90% identical to the amino acid sequence of FIG. 4A [SEQ ID NO:10] and/or 4B [SEQ ID NO:12] having endo-beta-N-acetylglucosamidase activity, also referred to as endo T derivatives or orthologs. Particular embodiments of the endo T derivatives or orthologs according to the invention relate to proteins, of which the amino acid sequence is at least 95% or particularly at least 98% identical to the protein sequence depicted in FIGS. 4A [SEQ ID NO:10] and/or 4B [SEQ ID NO:12], having endo-beta-N-acetylglucosamidase activity. Most particular embodiments of the invention relate to proteins having endo-beta-N-acetylglucosamidase activity of which the amino acid sequence corresponds to the sequence depicted in FIG. 4A [SEQ ID NO:10] or 4B [SEQ ID NO:12].

An endo T derivative or homologue having mannosyl-glycoprotein endo-beta-N-acetylglucosamidase activity refers to the fact that it demonstrates at least 50% conversion of substrate (i.e. endo-beta-N-acetylglucosamidase activity) as compared to the endo T isolated from T. reesei as can be assayed by the method described in the Examples section herein.

The invention further provides protein fragments of T. reesei Endo T (and DNA encoding for these fragments) which result from an N-terminal and/or C terminal truncation of the Endo T sequence depicted in FIG. 5a [SEQ ID NO:10] or 5b [SEQ ID NO:12] and which are catalytically active as can be determined by the assays described in the Examples section. Particular embodiments of the fragments according to the invention include but are not limited to a protein having the protein sequence from about amino acid 31 to about amino acid 310, a protein having the protein sequence from about amino acid 26 to about amino acid 316, a protein lacking the putative signal peptide (amino acid 1-17), a protein lacking the C-terminal sequence from about amino acid 317 onwards. A particular fragment is the 294 amino acid fragment (predicted Mr of 32,110) of T. reesei Endo T. depicted in FIG. 7 [SEQ ID NO:13].

According to a particular embodiment the proteins of the present invention are obtainable from T. reesei, and include isoforms of the Endo T protein disclosed in the present invention or can be naturally occurring variants, proteins derived from industrial strains of T. reesei and mutants generated by recombinant DNA technology (e.g. site directed mutatagenesis, transposon mediated mutagenesis), chemical mutagenesis or radiation.

The present invention further provides 5′ and 3′ UTR regions of T. reesei Endo T which allows the design of primers to amplify cDNA and genomic sequence of Endo T from wild-type T. reesei, natural and industrial strains of T. reesei and mutants generated by chemical mutagenesis or radiation.

A further aspect of the present invention relates to nucleotide sequences encoding a protein or a fragment thereof having endo-beta-N-acetylglucosamidase activity, which nucleotide sequence is at least 60%, more particularly at least 70%, most particularly at least 80%, especially at least 90%, identical to the nucleotide sequence depicted in FIGS. 3A [SEQ ID NO:7], 3B [SEQ ID NO:8], 4A [SEQ ID NO:9] and/or 4B [SEQ ID NO:11]. Particular embodiments of the invention relate to nucleotide sequences of which the sequence is at least 95%, or at least 98% identical to the DNA sequence depicted in FIGS. 3A [SEQ ID NO:7], 3B [SEQ ID NO:8], 4A [SEQ ID NO:9] and/or 4B[SEQ ID NO:11]. Most particular embodiments relate to nucleotide sequences encoding a protein or a fragment thereof having endo-beta-N-acetylglucosamidase activity, which nucleotide sequences correspond to the sequence depicted in FIGS. 3A [SEQ ID NO:7], 3B [SEQ ID NO:8], 4A [SEQ ID NO:9] and/or 4B [SEQ ID NO:11].

The present invention also discloses proteins and cDNA sequences encoding for proteins having a significant sequence similarity (i.e more than 60%, more than 70%, more than 80%, more than 85%, more than 90% similarity at the protein level in the common part of the sequence as obtained by the BLASTP algorithm without filter) which are or encode putative homologues of the T. reesei Endo T, i.e. proteins from other organisms having endo-beta-N-acetylglucosamidase activity.

Such proteins include but are not limited to proteins having the sequences identified as:

gb|EAA56225.1| hypothetical protein MG01876.4 Magnaporthe grisea . . .
ref|XP_{—}329440.1| predicted protein Neurospora crassa
gb|EAA75614.1| hypothetical protein FG05969.1 Gibberella zeae
gb|EM50314.1| hypothetical protein MG04073.4 Magnaporthe grisea
emb|CAD70866.1| related to chitinase Neurospora crassa
gb|EAA58983.1| hypothetical protein AN8245.2 Aspergillus niger
gb|AA088269.1| chitinase 3 Coccidioides immitis
ref|XP_{—}326886.1| predicted protein Neurospora crassa
gb|EAA69105.1| hypothetical protein FG02170.1 Gibberella zeae
or the cDNA and protein identifiable by EST clone gi/47730555 Metarhizium anisopliae

The invention further relates to the use of these proteins or derivatives or fragments thereof as endo-beta-N-acetylglucosamidases, such as, but not limited to in the production of biofuel.

Yet a further aspect of the present invention relates to the generation of recombinant proteins having endo-beta-N-acetylglucosamidase activity. The present invention discloses a cDNA sequence (FIGS. 3a [SEQ ID NO:7] and 3b [SEQ ID NO:8]) of T. reesei comprising an open reading frame (FIG. 4) [SEQ ID NO:9 and 11] encoding a protein (FIGS. 5a [SEQ ID NO:10] and 5b[SEQ ID NO:12]) with Endo T activity. The present invention thus discloses an Open Reading Frame (ORF) of Endo T with flanking 5′ and 3′ UTR DNA sequence which allow the generation of recombinant DNA molecules for overexpression of Endo T in T. reesei itself e.g. by placing the sequences of the invention under control of a strong promoter or for the expression of Endo T in other expression systems such as but not limited to other yeast expression systems such as Pichia, Saccharomyces or even in bacterial cells such as E. coli. Equally the enzyme can be cloned in insect or mammalian cells for the engineering of recombinant glycoproteins. The present invention also allows the generation of constructs for homologous recombination, wherein the complete Endo T gene or a part thereof is replaced by a selectable marker. Such constructs generate Endo T knockout strains, which have an increased glycosylation and an enhanced stability (of the organism and/or the secreted enzymes) which is advantageous for all applications wherein T. reesei is being used in bioreactors.

The present invention further also relates to deletion strains of a filamentous fungus. A deletion strain is a strain wherein the gene of interest is inactivated e.g. by the deletion of the gene via homologous recombination. Alternatively a yeast strain with an inactivated gene can also be generated by disruption of that gene (e.g the insertion of a foreign DNA seqeunce) or by the introduction of inactivating point mutations. Such deletion strains are of interest for the production of enzymes with an enhanced glycosylation and/or increased stability, due to the fact that the activity of a glycosidase enzyme is removed or reduced. Specific embodiments of this aspect of the invention relate to the production of cellulases with enhanced glycosylation and/or increased stability.

The present invention further also relates to vectors (eg cloning vectors or expression vectors) comprising DNA constructs expressing T. reesei Endo T or fragments thereof as a fusion protein with peptides or proteins for isolation (e.g. His Tag, Maltose binding protein, inteins, Gst) or identification (e.g. Green fluorescent protein).

Yet a further aspect of the present invention relates to methods for degrading biomass using the enzymes of the present invention. More particularly, the Endo T enzyme which is disclosed can be applied in the degradation of biomass (e.g. bio-fuel production) using organisms (e.g. recombinant bacteria or yeast) expressing Endo T or using a cultivation medium of such organisms comprising the secreted Endo T enzyme. Alternatively, the proteins having endo-beta-N-acetylglucosamidase activity of the invention are used directly in the in vitro production of ethanol from carbohydrate such as cellulose. Thus, according to a particular embodiment the sequence encoding Endo T of the invention or a fragment thereof having endo-beta-N-acetylglucosamidase activity is expressed on the surface of a yeast or bacterial strain. According to another particular embodiment of the invention, the simultaneous and synergistic saccharification and fermentation of amorphous cellulose to ethanol is ensured with only one recombinant yeast strain co-displaying different types of cellulolytic enzymes, including a protein having endo-beta-N-acetylglucosamidase according to the present invention. The present invention thus provides expression systems comprising a nucleotide sequence encoding a protein having endo-beta-N-acetylglucosamidase activity, more particularly a protein having at least 80% sequence identity with the amino acid sequence depicted in FIG. 4A or 5A [SEQ ID NO:10] and/or 4B or 5B [SEQ ID NO:12]. The isolation of T. reesei Endo T, the biochemical characterisation, the protein sequencing and deduction and determination of the cDNA encoding T. reesei is presented in the following examples.

### EXAMPLES

### Materials and Methods

Materials. Biogel P100 and molecular weight markers were purchased from Bio-Rad (Richmond, Calif.). Ultrafiltration membranes were purchased from Millipore corp. (Beford, Mass.).

### Microorganism and Culture Conditions.

T. reesei strain Rut-C30 was precultivated at 28° C. for 3 days in glucose (20 g/l) containing minimal medium (50 ml) and then induced for cellulase production with lactose (20 g/l) in corn steep liquor (Sigma) enriched media containing per litre: 5 g (NH₄)₂SO₄; 0.6 g CaCl₂; 0.6 g MgSO₄; 15 g KH₂PO₄; 15·10⁻⁴ g MnSO₄; 50·10⁻⁴ g FeSO₄.7H₂O; 20·10⁻⁴ g CoCl₂ en 15·10⁻⁴ g ZnSO₄. After 3 days, the extracellular medium is harvested and concentrated by diafiltration (Amicon® stirring cell) using a polyethersulfon membrane with a 10 kDa cut off (Millipore).

A 5-day, 14-litre fed-batch fermentation was set up by Iogen Corporation (Ottawa, Canada) using a rich medium with corn steep liquor as the nitrogen source. Temperature was maintained at 28° C. and pH at 4 (Hui et al., (2001) J. Chrom. B 752, 349-368). Samples were harvested 1, 3 and 5 days after the induction of cellulase production. Cultures of Endo T activity was assayed on filtered supernatant.

Assay of the Endo T activity. The Endo T activity was monitored/detected and quantified with FITC-labelled glycoprotein (RNAse B or Cel7A from T. reesei). Release of fluorescent deglycosylated protein was indicative of the Endo T activity present. One unit of activity is defined as the amount of enzyme necessary to transform 1 μmol of substrate per min. at 25° C. in 100 mM sodium acetate buffer pH 5.

SDS-PAGE. Proteins were separated by sodium dodecyl sulfate-polyacrylamide gel electrophoresis (SDS-PAGE) with 12.5% polyacrylamide gels stained with Coomassie blue.

Isoelectric focussing. Iso-electric focussing with Phast-Gel IEF 3-9 were also performed with a Phast System (Pharmacia). A dry precast homogeneous polyacrylamide gel (3.8 cm×3.3 cm) was rehydrated with 120 μl Pharmalyte™ 2.5-5 (Amersham Biosciences, Sweden), 20 μl Servalyt™ 3-7 (Serva Electrophoresis GmbH) and 1860 μl bidistilled water for two hours. In a prefocusing step (2000 V, 2.5 mA) the pH gradient was formed and 1 μl samples (10 mg protein/ml) were subsequently applied at the cathode position; electrophoresis was run to a final value of 450 Vh. Staining with Coomassie blue R-350 was according to the manufacturer's instructions. Amyloglucosidase (IP 3.5), methyl red (dye, IP 3.75), soybean trypsin inhibitor (IP 4.55), lactoglobulin A (IP 5.2) and bovine carbonic anhydrase (IP 5.85) (Amersham Biosciences, Sweden) were used as marker proteins.

Electrospray ionisation mass spectrometry. Mass spectra were acquired on a Q-TOF instrument (Micromass, UK) equipped with a nanospray source. The samples were desalted using an Ultrafree™-filter, MWCO 10 kDa (Millipore), dissolved in 50% acetonitrile (0.1% formic acid) to a final concentration of 5 pmol/μl, and measured in the positive mode (needle voltage +1250 V) using Protana (Odense, UK) needles. Mass spectra were processed using MaxEnt software. Mass accuracy was typically within 0.01-0.02% from the calculated value.

### Determination of Internal Peptide Sequences.

Peptide fragments were determined as described in Samyn et al., (2004) J. of the Am. Soc. Mass 15, 1838-1852.

Cloning of T. reesei Endo T Sequence.

PCR amplification with genomic DNA of T. reesei as a template was amplified with a proofreading DNA polymerase using forward primer 5′ gatgaaggcgtccgtctacttg 3′ [SEQ ID NO:14] and reverse primer 5′ cgcccttatactctttgcctatttc 3′ [SEQ ID NO:15]. A fragment of about 1100 bp was isolated from agarose gel and cloned into a vector. Three independent clones were sequenced.

### Example 1

### Production of Endo T Using T. reseei

T. reesei was grown in corn steep liquor enriched medium as described (Hui et al., (2001) J. Chrom. B 752, 349-368). Endo T activity was monitored on filtered supernatant from growing cells. Endo T Activity was present from the beginning of the cultivation. Because of the low production of Endo T activity in the medium (2.51 mU/ml), culture growth was stopped just before the secretion of cellulases. Endo T is an enzyme found in the culture medium and not in the cells, indicating that Endo T is secreted.

### Example 2

### Purification of Endo T and Characterization

Using Man₅GlcNAc₂-RNase B as substrate, the endo-D-N-acetylglucosaminidase was purified 1300-fold from the culture medium of T. reesei (Table 1). The Avicel adsorption step was efficient in removing CBM containing proteins (cellulases) and facilitated the subsequent purification but resulted in a substantial loss of activity (61%, see Table 4). This is probably due to affinity of the Endo T protein for the glycosylated cellulases bound to Avicel. However, an 14-fold enrichment was obtained during this first purification step. The non-bound fraction was applied to a DEAE-sepharose-FF column (10×1 cm), which was subsequently eluted with a linear gradient of 5 mM NH₄OAc to 300 mM NH₄OAc, pH 5. Proteins were monitored at 280 nm, and the Endo T activity was assayed with the FITC-labelled glycoproteins (data not shown). The purification is also monitored by activity measurements on invertase (10 μl of the fractions were incubated with 10 μl 10 mg/ml substrate dissolved in 100 mM sodium acetate buffer pH 5). Activity is followed by 7.5% SDS-PAGE. The enzyme activity eluted at high acetate concentration and was pooled. This purification step resulted in a substantial enrichment (172 fold) and almost no loss of activity (Table 1).

The enzyme fraction was dialyzed and applied to the Biogel column. The purification is monitored by classical band shifting using invertase. After this step, the enzyme was purified about 1300 fold from culture medium with a yield of 25% (Table 1). Endo T was concentrated to about 1000 μl. By using p-nitrophenyl glycosides as the substrate, the enzyme preparation was found to contain no exoglycosidases. The purified Endo T preparation showed a double protein band on SDS-polyacrylamide gels (FIG. 1, lane 5); and the molecular mass was estimated to be 30 kDa under reducing conditions. PAS staining proved the protein to be non-glycosylated, although four potential N-glycosylation sites are present according to the deduced protein sequence.

TABLE 1
Purification of Endo T from the culture filtrate of T. reesei

| | | | | Specific | | |
|---|---|---|---|---|---|---|
| | | Protein | Activity | activity | Yield | Enrichment |
| Purification step | | (mg) | (U) | (mU/mg) | (%) | factor |
| 1 | Culture | 4500 | 0.753 | 0.17 | 100 | 1 |
| | filtrate | | | | | |
| 2 | Adsorption | 125 | 0.291 | 2.3 | 39 | 14 |
| 3 | DEAE- | 9.5 | 0.273 | 29 | 36 | 172 |
| | sepaharose | | | | | |
| 4 | Biogel P100 | 0.87 | 0.192 | 220 | 25 | 1318 |

The specific activity of Endo T (220 mU/mg) is lower than that of Streptomyces plicatus Endo H, (5200 mU/mg) as measured with the quantitative method at 25° C., pH 5.

Electrospray ionisation mass spectrometry Experiments with the purified protein indicated a theoretical Mr of 31 775 and 32 102.

Aminoterminal sequence determination of the major band on SDS page (AEPTDLP . . . ) [SEQ ID NO:16] indicates that the mature protein starts at position 27 (numbering of FIG. 7).

The Mr of 32102 indicates that the mature protein has a length of 294 amino acids as depicted in FIG. 7. Assuming that the minor band on SDS page has the same aminoterminal sequence, this band could corresponds with protein of 291 with the sequence . . . PGLVPEL [SEQ ID NO: 17] at the carboxyterminus

### Example 3

### Identification of the Protein and cDNA Sequence of T. reesei Endo T

### a) Sequence Information Obtained by Enzymatic and Chemical Fragmentation of the Protein

Internal peptide sequences of Endo T were determined by enzymatic and chemical fragmentation and MS identification. The most informative results are depicted in Table 2.

TABLE 2

Partial sequence information of T. reesei
Endo T obtained by digestion under
different conditions

| | |
|---|---|
| Mass (Da) | Sequence |

| | | | |
|---|---|---|---|
| A | 2099.92 | TIDSPDSATFEHYY | [SEQ ID NO: 18] |
| | 2948.32 | D......DIDVEQXXSQQGIDR | [SEQ ID NO: 19] |
| B | 1082.00 | AEPTD | [SEQ ID NO: 20] |
| | 1306.33 | EIIR | [SEQ ID NO: 21] |
| | 2283.88 | TIDSPDSATFEHYYXXXR | [SEQ ID NO: 22] |
| | 3155.22 | DAIVNFXXXXXXIDVEQXXXQQ | [SEQ ID NO: 23] |
| | | GIDR | |
| C | 2079.11 | | |
| | 3186.63 | ......DSPDSATXX..... | [SEQ ID NO: 24] |
| | 3212.34 | VGGAAPGSFNTQTIDSPDSATF | [SEQ ID NO: 25] |
| | | EHYY... | |
| | 3230 = 32 | .......TIDSPDSATFEH... | [SEQ ID NO: 26] |

A. Trypsin digest: Peptides and MS/MS fragmentation data obtained after guanidinylation.
B. Trypsin digest: Peptides and MS/MS fragmentation data obtained after guanidinylation and sulfonylation.
C. CNBr-digest and subsequent trypsine treatment: Peptides and MS/MS fragmentation data obtained after guanidinylation.

An overview of all peptide sequence data obtained is provided in tables 3 to 8 hereunder.

TABLE 3

peptide sequences after trypsin digest and
guanidinylation

| Determined | Theoretical | Experimental |
|---|---|---|
| Mass (Da) | sequence | sequence |
| 2099.9207 | TIDSPDSATFEHYYG | TIDSPDSATFEHYY |
| | QIR | |
| | [SEQ ID NO: 27] | [SEQ ID NO: 18] |
| 2948.3289 + | DAIVNFQLEGMDIDV | D.........DIDVE |
| 2 × oxidated | EQPMSQQGIDR | QXXSQQGIDR |
| | [SEQ ID NO: 28] | [SEQ ID NO: 19] |

TABLE 4

peptide sequences after trypsin digest and
sulfonylation

| Determined | Theoretical | Experimental |
|---|---|---|
| Mass (Da) | sequence | sequence |
| 1082.00 | AEPTDLPR | AEPTD |
| | [SEQ ID NO: 29][SEQ ID NO: 20] | |
| | EILRPGLVPE | EIIR |
| | [SEQ ID NO: 30] | [SEQ ID NO: 21] |
| 1817.40 | | Several small |
| | | peaks |
| 2283.88 | TIDSPDSATFEHYYG | TIDSPDSATFEHYYX |
| | QIR | XXR |
| | [SEQ ID NO: 27] | [SEQ ID NO: 22] |
| 3155.22 + | DAIVNFQLEGM_{OX}DI | DAIVNFXXXXXXIDV |
| 1 × oxidation | DVEQPMSQQGIDR | EQXXXQQGIDR |
| (3148) | [SEQ ID NO: 28] | [SEQ ID NO: 23] |

TABLE 5
peptide sequences after Glu-C digest

| | | |
|---|---|---|
| Determined | Theoretical | Experimental |
| Mass (Da) | sequence | sequence |

| | | |
|---|---|---|
| 898.33 | AEPTDLPR | XXXXDIPR |
| | [SEQ ID NO: 29] | [SEQ ID NO: 31] |
| 936.34 | HYYGQLR | .....R |
| | [SEQ ID NO: 32] | |
| 993.47 | ILRPGLVPE | |
| | [SEQ ID NO: 33] | |
| 1918.60 | GMDIDVEQPMSQQIDR | XXDIDVEQ |
| | [SEQ ID NO: 34] | [SEQ ID NO: 35] |
| 1934.60 | GMOXDIDVEQPMSQQ | |
| | IDR | |
| | [SEQ ID NO: 34] | |

TABLE 6

Peptide sequence results of peptides
obtained after CNBr fragmentation of
Endo T.

| | | |
|---|---|---|
| Determined | Theoretical | Experimental |
| Mass (Da) | sequence | sequence |

| | | |
|---|---|---|
| 812 | KQAGVKVM | QQAGVQVM |
| | [SEQ ID NO: 36] | [SEQ ID NO: 37] |
| 2940.44 | AEPTDLPRLIVYFQT | .....D.....QTTH |
| | THDSSNRPISM | DSS.......... |
| | [SEQ ID NO: 38] | [SEQ ID NO: 39] |
| 4355 | VGGAAPGSFNTQTLD | |
| | SPDSATFEHYYGQLR | |
| | DAIVNFQLEGM | |
| | [SEQ ID NO: 40] | |

TABLE 7

peptide sequence results and Mw (Mr) of
peptides obtained after CNBr fragmentation,
followed by enzymatic digest with trypsin,
of Endo T.

| | | |
|---|---|---|
| Determined Theoretical | Experimental | |
| Mass (Da) | sequence | sequence |

| | | |
|---|---|---|
| 2079.11 | LIVYFQTTHDSSNRP | |
| | ISM | |
| | [SEQ ID NO: 41] | |
| 3186.6389 | VGGAAPGSFNTQTLD | ......DSPDSA |
| | SPDSATFEHYYGQLR | TXX..... |
| | [SEQ ID NO: 42] | [SEQ ID NO: 24] |
| 3212.3394 = | VGGAAPGSFNTQTLD | VGGAAPGSFNTQTID |
| 3186.6389 + | SPDSATFEHYYGQLR | SPDSATFEHYY... |
| ? | [SEQ ID NO: 42] | [SEQ ID NO: 25] |
| 3230 = | VGGAAPGSFNTQTLD | TIDSPDSATFEH... |
| 3186.6289 + | SPDSATFEHYYGQLR | [SEQ ID NO: 26] |
| + ? | [SEQ ID NO: 42] | |
| 987.551 | IVANGFAPAK | ....ANGFA... |
| | [SEQ ID NO: 43] | [SEQ ID NO: 44] |
| 1689.87 Da | GSLQDGQFVAAEPDG | VAAE |
| | AK | [SEQ ID NO:54] |
| | [SEQ ID NO: 45] = | |
| | RIBONUCLEASE | |
| | Tkv | |
| 1700.87 | DIDVEQPMSQQIDR | DIDVEQPMXXXXXDR |
| | [SEQ ID NO: 46] | [SEQ ID NO: 47] |
| 2079.11 | LIVYFQTTHDSSNRP | ...YFQTTHDSSNR.... |
| | ISM | [SEQ ID NO: 48] |
| | [SEQ ID NO: 41] | |
| 3212.3394 | VGGAAPGSFNTQTLD | XXGAAPGSFNTQTID |
| =3186.6389 | SPDSATFEHYYGQLR | SPDSATFEHYYXXXR |
| + ? | [SEQ ID NO: 42] | [SEQ ID NO: 49] |
| 3230 = | VGGAAPGSFNTQTLD | ........TIDSPDS |
| 3186.6289 + | SPDSATFEHYYGQLR | ATFEH... |
| ? | [SEQ ID NO: 42] | [SEQ ID NO: 26] |

TABLE 8

peptide sequence results and Mr of
peptides of Endo T, obtained after CNBr
fragmentation, followed by enzymatic digest
with Glu-C.

| | | |
|---|---|---|
| Determined | Theoretical | Experimental |
| Mass (Da) | sequence | sequence |

| | | |
|---|---|---|
| 993.633 | IIRPGLVPE | II......PE |
| | [SEQ ID NO: 50] | |
| 1590.8 | | Several peaks |
| 1966 | | YWH....DDGE |
| | | [SEQ ID NO: 51] |
| 2269.54 | VGGAAPGSFNTQTL | ....SDPSD... |
| | DSPDSATFE | [SEQ ID NO: 53] |
| | [SEQ ID NO:52] | |
| 2906.56 | | Several peaks |

b) Screening of Protein and cDNA Databases

The most informative peptide sequences were used to screen sequence databases using the BLAST facility at the NCBI website. No significant sequence similarity was found with complete protein or cDNA sequences (NR database). However, using the TBLASTN algorithm and the EST database, several clones of T. reesei were encountered which encode peptide sequences identical to the experimentally determined peptide sequences of Endo T. depicted in Table 2-8.

For example, the peptide VGGAAPGSFNTQTIDSPDSATFEHYY [SEQ ID NO:25] is encoded by EST clones with GI numbers 30122409, 38135670, 38138150, 38120437, 30124281, 30110396 (Foreman et al., (2003) J. Biol. Chem. 278, 31988-31997; Diener et al., (2004) FEMS Microbiol. Lett. 230, 275-282).

### c) Screening of an EST Database

Using the clones obtained under (b) themselves as probes for screening the EST database (BLASTN algorithm) a set of overlapping clones was identified. These cDNA sequences were trimmed to remove non-informative sequences (stretches of unidentified nucleotides N).

While constructing the alignment it became evident that a number these EST sequences were likely to be sequences which were submitted twice as they contain the same irregularities. An alignment of a non-redundant set of EST sequences [SEQ ID NO:1 to 6] is depicted in FIG. 2. This alignment gives, for the majority of the sequence, at least a two-fold confirmation of the sequence which allows the determination of a consensus sequence. At the 3′ end the alignment provides a two-fold confirmation of the sequence. For this part the sequence with the least ambiguities was preferred.

The consensus-sequence [SEQ ID NO:7] which was derived from this alignment was screened for the presence of an open reading frame using the ORF Finder algorithm at the NCBI website.

This reveals the presence of an open reading frame encoding a protein of 359 amino acids. The protein sequence has a predicted signal sequence MKASVYLASLLATLSMA [SEQ ID NO:55].

Assuming an average Mr of 110 for an amino-acid, the theoretical Mr of Endo T is about 39000 or 35000, which is seemingly in disagreement with the Mr detected by Mass spectrometry. This suggests that the protein is further proteolytically processed in the yeast or upon secretion by the yeast in the medium. Alternatively it indicates that the protein is susceptible to proteolytic degradation during cultivation and/or purification.

Evidence for processing or degradation at both N-terminal and C-terminal is derived from FIG. 6 wherein the experimentally determined peptide sequences are indicated on the amino acid sequence of T. reesei Endo T. The protein which has been isolated comprises at least the sequence from amino acids 26 up to amino acid 316 [SEQ ID NO:13]. Such a protein has a calculated Mr of 31674 which approximates the values determined by Mass spectrometry.

The relevance of the N-terminal sequence from amino acid 1 to 26 and the C terminal sequence from amino acid 317 to 359 can be evaluated by the generation of recombinant truncated molecules at either the N terminus, C terminus or both.

### Example 4

### Designing of Primers for the Cloning of the Endo T Sequence

Based upon the sequence depicted in FIG. 3 primers were generate in the 5′ and 3′ UTR sequence for PCR amplification of Endo T. These primers are in the first instance used to amplify the sequence of Endo T of T. reesei and to confirm or correct the ORF encoding Endo T:

[SEQ ID NO: 56]

| | |
|---|---|
| Forward primer: | 5′-ctgtaaagaggcttcaccccg-3′ |

[SEQ ID NO: 57]

| | |
|---|---|
| Reverse primer: | 5′-ttcatgctctcatcacacag-3′ |

Also the sequence as depicted in FIG. 4 allows the generation of primers to clone Endo T in cloning or expression vectors, e.g.:

forward primer:
(EcoRV, NdeI)

[SEQ ID NO: 58]

5′-ggggatatcatatgaaggcgtccgtctacttggcg-3′
reverse primer:
(EcoRV, XbaI)

[SEQ ID NO: 59]

5′-ggggatatctagataaagcattcaccatagcataatag-3′

Equally the sequence of FIG. 4 [SEQ ID NO:9] allows the generation of primers for the sequencing of Endo T, suitable to verify the sequence of the ORF derived by the assembly of the EST sequences or for the sequence determination of mutant Endo T sequences. Exemplary primers in addition to the above ones are:

| | |
|---|---|
| 5′-acgcacctcattgtgtgctcg-3′ | [SEQ ID NO: 60] |
| 5′-gtgggcggcgcggcgccgggg-3′ | [SEQ ID NO: 61] |
| 5′-gaggatagcagcaacctgtcc-3′ | [SEQ ID NO: 62] |
| 5′-ctcgtgagcgagtacggccag-3′ | [SEQ ID NO: 63] |
| 5′-gaggagagcgtcaaggcg-3′ | [SEQ ID NO: 64] |

### Example 5

### Cloning of T. reesei Endo T

Using the above primers, T. reesei Endo T was amplified from genomic DNA. The amplified product was sequenced. This DNA sequence is depicted in FIG. 2 in the bottom line of the alignment and also in FIG. 3B [SEQ ID NO:8]. The translation product of this experimental DNA sequence [SEQ ID NO:12] is depicted in FIG. 4b, 5b and in the bottom line of the sequence alignment of FIG. 5c.

Six differences in the coding region are present between the EST assembled sequence and the cloned sequence to 4 differences at the amino acid level. The sequences are 99% identical at the protein level. The first difference (Gly instead of Glu) is located in the amino terminal region, which is cleaved off. Two other changes in the amino acid sequence (Thr/Ala at position 253, and Gly/Ser at position 319) are located at places, which were not confirmed by mass spectrometry. Both deal with substitutions having little impact on the physicochemical properties of the side chains.

Finally, one amino acid difference (Lys (alkaline) instead of Glu (acidic)), at position 307 is in contradiction with both the mass spectrometry data and the in silico assembled sequence.

1-20. (canceled)

21. An isolated polynucleotide encoding a protein of a filamentous fungus or a fragment thereof, said protein having an amino acid sequence as depicted in FIG. 5A [SEQ ID NO:10] or 5B [SEQ ID NO:12], or a sequence having at least 70% sequence similarity therewith, said protein or protein fragment having mannosyl-glycoprotein endo-beta-N-acetylglucosaminidase activity.

22. The isolated polynucleotide according to claim 21 comprising a nucleotide sequence encoding the putative glycoside hydrolase 18 domain sequence indicated in FIG. 5A.

23. The isolated polynucleotide according to claim 21 comprising the nucleotide sequence depicted in FIG. 4A [SEQ ID NO:9] or 4B [SEQ ID NO:11] or a sequence with at least 70% sequence identity therewith.

24. The isolated polynucleotide according to claim 21, wherein said filamentous fungus is Trichoderma sp.

25. A method for the expression of a protein or protein fragment having mannosyl-glycoprotein endo-beta-N-acetylglucosaminidase activity, comprising introducing an isolated polynucleotide encoding a protein having an amino acid sequence as depicted in FIG. 5A [SEQ ID NO:10] or 5B [SEQ ID NO:12], or a sequence having at least 70% sequence similarity therewith or encoding a fragment of said protein, said protein or protein fragment having mannosyl-glycoprotein endo-beta-N-acetylglucosaminidase activity, in a suitable host and ensuring expression thereof.

26. An isolated polypeptide of a filamentous fungus, having mannosyl-glycoprotein endo-beta-N-acetylglucosaminidase activity, having an amino acid sequence as depicted in FIG. 5A [SEQ ID NO:10] or 4B [SEQ ID NO:12] or an amino acid sequence with at least 70% sequence similarity to the amino acid sequence depicted in FIG. 5A [SEQ ID NO:10] or 4B [SEQ ID NO:12] or a fragment thereof with mannosyl-glycoprotein endo-beta-N-acetylglucosaminidase activity.

27. The isolated polypeptide according to claim 26 wherein said fragment comprises the putative glycoside hydrolase 18 domain sequence indicated in FIG. 5A.

28. The isolated polypeptide according to claim 26, which is a fragment of the sequence as depicted in FIG. 5A [SEQ ID NO:10] or 4B [SEQ ID NO:12], wherein said sequence has been N terminally and/or C terminally truncated.

29. A method for the degradation of organic material comprising producing a polypeptide according to claim 26, and contacting said polypeptide with organic material, thereby degrading said organic material.

30. The method according to claim 29, wherein said degradation is performed in a medium with a pH between 4.5 and 5.5.

31. A method for the production of an enzyme with an enhanced glycosylation and/or increased stability, comprising culturing an Endo T deletion strain of a filamentous fungus and ensuring expression of said enzyme.

32. The method according to claim 31, wherein said enzyme is a cellulase.

33. An antibody directed against the polypeptide of claim 26.

34. A process for the production of bio-fuel, said process comprising the steps of degrading organic material with a polypeptide according to claim 26 and recovering the degraded organic material.

35. A transgenic cell comprising a foreign DNA comprising the polynucleotide of claim 21.

36. A yeast cell comprising in its genome the nucleotide sequence of claim 21, under control of a foreign promoter.

37. An endo-beta-N-acetylglucosaminidase deletion strain of a filamentous fungus, wherein a gene encoding a protein of a filamentous fungus, having an amino acid sequence as depicted in FIG. 5A [SEQ ID NO:10] or 5B [SEQ ID NO:12], or a sequence having at least 70% sequence similarity therewith having mannosyl-glycoprotein endo-beta-N-acetylglucosaminidase activity, is inactivated.

38. The deletion strain according to claim 37, wherein the filamentous fungus is T. reesei.

39. The process for the production of bio-fuel according to claim 34, wherein said polypeptide is obtained by introducing into a micro-organism a sequence encoding a protein having endo-beta-N-acetylglucosaminidase activity, said protein having a sequence with at least 70% sequence identity to the amino acid sequence depicted in FIG. 5A [SEQ ID NO:10] or 5B [SEQ ID NO:12] and ensuring over-expression of said protein in said micro-organism.

40. The process of claim 39, wherein said micro-organism is a yeast or bacterial cell.


SEQUENCE LISTING (64 sequences)

SEQ ID NO: 1 - DNA, 719 bp, Trichoderma reesei
  misc_feature (1)..(719): EST clone
cccacgcgtccgacttggtgtccctgctggcgacgctgtcgatggcggtgcccgtcaaggagctgcagctgcgggccgagccgacggacctgcctcgcctgattgtgtacttccagacgacgcacgacagcagcaaccggcccatctcgatgctgccgctcatcacggagaagggcatcgcgctgacgcacctcattgtgtgctcgttccacatcaaccaaggcggcgtggtgcacctcaacgacttcccgccggacgacccgcacttctacacgctgtggaacgagactatcacgatgaagcaggcgggcgtcaaggtcatgggcatggtgggcggcgcggcgccggggtcctttaacacgcagacgctcgactcgccggactcggccacgtttgagcactactacgggcagctgagggacgccattgtcaacttccagctcgagggcatggacctggacgtcgagcagccgatgagccagcagggcatcgaccggctgattgcgcggctgcgggcggatttcgggcccgactttctcatcacgctggcgcccgtcgcgtcggcgctcgaggatagcagcaacctgtccggcttcagctacacggcgctgcagcagacgcagggcaacgacattgactggtacaacacgcagttctacagcggctttggcagcatggcggacacgagcgactacgaccgcatcgtggccaacggntcc

SEQ ID NO: 2 - DNA, 755 bp, Trichoderma reesei
  misc_feature (1)..(755): EST clone
nttccttttttangcgctgnctgtcactagccctntgttaaagggcctaccggtcgacccacgcgtccggccgagccgacggacctgcctcgcctgattgtgtacttccagacgacgcacgacagcagcaaccggcccatctcgatgctgccgctcatcacggagaagggcatcgcgctgacgcacctcattgtgtgctcgttccacatcaaccaaggcggcgtggtgcacctcaacgactttccgtcggacgacccgcacttctacacgctgtggaacgagactatcacgatgaagcaagcgggcgtcaaggtcatgggcatgtgggcggcgcggcgccggggtccttttacacgcagacgctcgactcgccggactcgggcacgtttgagcactactacgggcagctgagggacgccattgtcaacttccagctcgagggcatggacctggacgtcgagcagccgatgagccagcagggcatcgaccggctgattgcgcggctgcgggcggatttcgggcccgacttcctcatcacgctggcgcccgtcgcgtcggcgctcgaggatagcagcaacctgtccggcttcagctacacggcgctgcagcagacgcagggcaacgacattgactggtacaacacgcagttctacagcggcttcggcagcatggcggacacgagcgactacgaccgcatcgtggccaacgggttcgcgcccgccaaggtggtggccggccagctgacgacgcccgag

SEQ ID NO: 3 - DNA, 714 bp, Trichoderma reesei
  misc_feature (1)..(755): EST clone
ctgtaagaggcttcacctcgtctcttcttttctgacttgctccctgcccttgccccccctcctccgaccccctccgcctcccccctcctttgttcacgatgaaggcgtccgtctacttggcgtccctgctggcgacgctgtcgatggcggtgcccgtcaaggagctgcagctgcgggccgagccgacggacctgcctcgcctgattgtgtacttccagacgacgcacgacagcagcaaccggcccatctcgatgctgccgctcatcacggagaagggcatcgcgctgacgcacctcattgtgtgctcgttccacatcaaccaaggcggcgtggtgcacctcaacgacttcccgccggacgacccgcacttctacacgctgtggaacgagactatcacgatgaagcaggcgggcgtcaaggtcatgggcatggtgggcggcgcggcgccggggtcctttaacacgcagacgctcgactcgccggactcggccacgtttgagcactactacgggcagctgagggacgccattgtcaacttccagctcgagggcatggacctggacgtcgagcagccgatgagccagcagggcatcgaccggctgattgcgcggctgcgggcggatttcgggcccgacttcctcatcacgctggcgcccgtcgcgtcggcgctcgaggatagcagcaacctgttcggctttagctacacggcgctga

SEQ ID NO: 4 - DNA, 731 bp, Trichoderma reesei
  misc_feature (1)..(731): EST clone
cccacgcgtccgggatatgtatcgtcctgtaagaggcttcaccccgtctcttcttttctgacttgctccctgcccttgccccccctcctccgaccccctccgcctcccccctcctttgttcacgatgaaggcgtccgtctacttggcgtccctgctggcgacgctgtcgatggcggtgcccgtcaaggagctgcagctgcgggccgagccgacggacctgcctcgcctgattgtgtacttccagacgacgcacgacagcagcaaccggcccatctcgatgctgccgctcatcacggagaagggcatcgcgctgacgcacctcattgtgtgctcgttccacatcaaccaaggcggcgtggtgcacctcaacgacttcccgccggacgacccgcacttctacacgctgtggaacgagactatcacgatgaagcaggcgggcgtcaaggtcatgggcatggtgggcggcgcggcgccggggtcctttaacacgcagacgctcgactcgccggactcggccacgtttgagcactactacgggcagctgagggacgccattgtcaacttccagctcgagggcatggacctggacgtcgagcagccgatgagccagcagggcatcgaccggctgattgcgcggctgcgggcggatttcgggcccgacttcctcatcacgctggcgcccgtcgcgtcggcgctcgaggatagcagcaacctgtccggcttcagctacc

SEQ ID NO: 5 - DNA, 729 bp, Trichoderma reesei
  misc_feature (1)..(729): EST clone
gccattgtcaacttccagctcgagggcatggacctggacgtcgagcagccgatgagccagcagggcatcgaccggctgattgcgcggctgcgggcggatttcgggcccgacttcctcatcacgctggcgcccgtcgcgtcggcgctcgaggatagcagcaacctgtccggcttcagctacacggcgctgcagcagacgcagggcaacgacattgactggtacaacacgcagttctacagcggcttcggcagcatggcggacacgagcgactacgaccgcatcgtggccaacgggttcgcgcccgccaaggtggtggccggccagctgacgacgcccgagggcgcgggctggatcccgacgagcagcctcaacaacaccattgtctcgctcgtgagcgagtacggccagattggcggcgtcatgggctgggagtacttcaacagcctgcccggcggcaccgcggagccgtgggagtgggcgcagattgtgacggagattctgaggccgggcttggtgccggagctgaagattacggaggacgatgcggcgaggctgacgggtgcgtatgaggagagcgtcaaggcggcggcggcggacaacaagagctttgtgaagaggcctagcattaactattatgctatggtgatgnctttaagggnaggngggacanaggggggaaataggcaaagagtataaggggcggttttgtatataggctgtgtgatgaan

SEQ ID NO: 6 - DNA, 555 bp, Trichoderma reesei
  misc_feature (1)..(555): EST clone
cattgactggtacaacacgcagttctacagcggcttcggcagcatggcggacacgagcgactacgaccgcatcgtggccaacgggttcgcgcccgccaaggtggtggccggccagctgacgacgcccgagggcgcgggctggatcccgacgagcagcctcaacaacaccattgtttcgctcgtgagcgagtacggccagattggcggcgtcatgggctgggagtacttcaacagcctgcccggcggcaccgcggagccgtgggagtgggcgcagattgtgacggagattttgaggccgggcttggtgccggagctgaagattacggaggacgatgcggcgaggctgacgggtgcgtatgaggagagcgtcaaggcggcggcggcggacaacaagagctttgtgaagaggcctagcattaactattatgctatggtgaatgcttaagggaggggggacaaaggggggaaataggcaaagagtataagggcggtttttgtatataggctgtgtgatgagagcatgaattgatattcagtattgtgttaacaaacttg

SEQ ID NO: 7 - DNA, 1290 bp, Trichoderma reesei
ctgtaagaggcttcaccccgtctcttcttttctgacttgctccctgcccttgccccccctcctccgaccccctccgcctcccccctcctttgttcacgatgaaggcgtccgtctacttggcgtccctgctggcgacgctgtcgatggcggtgcccgtcaaggagctgcagctgcgggccgagccgacggacctgcctcgcctgattgtgtacttccagacgacgcacgacagcagcaaccggcccatctcgatgctgccgctcatcacggagaagggcatcgcgctgacgcacctcattgtgtgctcgttccacatcaaccaaggcggcgtggtgcacctcaacgacttcccgccggacgacccgcacttctacacgctgtggaacgagactatcacgatgaagcaggcgggcgtcaaggtcatgggcatggtgggcggcgcggcgccggggtcctttaacacgcagacgctcgactcgccggactcggccacgtttgagcactactacgggcagctgagggacgccattgtcaacttccagctcgagggcatggacctggacgtcgagcagccgatgagccagcagggcatcgaccggctgattgcgcggctgcgggcggatttcgggcccgacttcctcatcacgctggcgcccgtcgcgtcggcgctcgaggatagcagcaacctgtccggcttcagctacacggcgctgcagcagacgcagggcaacgacattgactggtacaacacgcagttctacagcggcttcggcagcatggcggacacgagcgactacgaccgcatcgtggccaacgggttcgcgcccgccaaggtggtggccggccagctgacgacgcccgagggcgcgggctggatcccgacgagcagcctcaacaacaccattgtttcgctcgtgagcgagtacggccagattggcggcgtcatgggctgggagtacttcaacagcctgcccggcggcaccgcggagccgtgggagtgggcgcagattgtgacggagattttgaggccgggcttggtgccggagctgaagattacggaggacgatgcggcgaggctgacgggtgcgtatgaggagagcgtcaaggcggcggcggcggacaacaagagctttgtgaagaggcctagcattaactattatgctatggtgaatgcttaaaggggaggggggacaaaggggggaaataggcaaagagtataagggcggtttttgtatataggctgtgtgatgagagcatgaattgatattcagtattgtgttaacaaacttg

SEQ ID NO: 8 - DNA, 1126 bp, Trichoderma reesei
gatgaaggcgtccgtctacttggcgtccctgctggcgacgctgtcgatggcggtgcccgtcaaggggctgcagctgcgggccgagccgacggacctgcctcgcctgattgtgtacttccagacgacgcacgacagcagcaaccggcccatctcgatgctgccgctcatcacggagaagggcatcgcgctgacgcacctcattgtgtgctcgttccacatcaaccaaggcggcgtggtgcacctcaacgacttcccgccggacgacccgcacttctacacgctgtggaacgagactatcacgatgaagcaggcgggcgtcaaggtcatgggcatggtgggcggcgcggcgccggggtcctttaacacgcagacgctcgactcgccggactcggccacgtttgagcactactacgggcagctgagggacgccattgtcaacttccagctcgagggcatggacctggacgtcgagcagccgatgagccagcagggcatcgaccggctgattgcgcggctgcgggcggatttcgggcccgacttcctcatcacgctggcgcccgtcgcgtcggcgctcgaggatagcagcaacctgtccggcttcagctacacggcgctgcagcagacgcagggcaacgacattgactggtacaacacgcagttctacagcggcttcggcagcatggcggacacgagcgactacgaccgcatcgtggccaacgggttcgcgcccgccaaggtggtggccggccagctgacggcgcccgagggcgcgggctggatcccgacgagcagcctcaacaacaccattgtctcgctcgtgagcgagtacggccagattggcggcgtcatgggctgggagtacttcaacagcctgcccggcggcaccgcggagccgtgggagtgggcgcagattgtgacgaagattctgaggccgggcttggtgccggagctgaagattacggaggacgatgcggcgaggctgacgagtgcgtatgaggagagcgtcaaggcggcggcggcggacaacaagagctttgtgaagaggcctagcattaactattatgctatggtgaatgcttaagggaggggggacaaaggggggaaataggcaaagagtataagggcg

SEQ ID NO: 9 - DNA, 1080 bp, Trichoderma reesei
  CDS (1)..(1080)
atgaaggcgtccgtctacttggcgtccctgctggcgacgctgtcgatggcggtgcccgtcaaggagctgcagctgcgggccgagccgacggacctgcctcgcctgattgtgtacttccagacgacgcacgacagcagcaaccggcccatctcgatgctgccgctcatcacggagaagggcatcgcgctgacgcacctcattgtgtgctcgttccacatcaaccaaggcggcgtggtgcacctcaacgacttcccgccggacgacccgcacttctacacgctgtggaacgagactatcacgatgaagcaggcgggcgtcaaggtcatgggcatggtgggcggcgcggcgccggggtcctttaacacgcagacgctcgactcgccggactcggccacgtttgagcactactacgggcagctgagggacgccattgtcaacttccagctcgagggcatggacctggacgtcgagcagccgatgagccagcagggcatcgaccggctgattgcgcggctgcgggcggatttcgggcccgacttcctcatcacgctggcgcccgtcgcgtcggcgctcgaggatagcagcaacctgtccggcttcagctacacggcgctgcagcagacgcagggcaacgacattgactggtacaacacgcagttctacagcggcttcggcagcatggcggacacgagcgactacgaccgcatcgtggccaacgggttcgcgcccgccaaggtggtggccggccagctgacgacgcccgagggcgcgggctggatcccgacgagcagcctcaacaacaccattgtttcgctcgtgagcgagtacggccagattggcggcgtcatgggctgggagtacttcaacagcctgcccggcggcaccgcggagccgtgggagtgggcgcagattgtgacggagattttgaggccgggcttggtgccggagctgaagattacggaggacgatgcggcgaggctgacgggtgcgtatgaggagagcgtcaaggcggcggcggcggacaacaagagctttgtgaagaggcctagcattaactattatgctatggtgaatgcttaa

SEQ ID NO: 10 - protein, 359 aa, Trichoderma reesei
MKASVYLASLLATLSMAVPVKELQLRAEPTDLPRLIVYFQTTHDSSNRPISMLPLITEKGIALTHLIVCSFHINQGGVVHLNDFPPDDPHFYTLWNETITMKQAGVKVMGMVGGAAPGSFNTQTLDSPDSATFEHYYGQLRDAIVNFQLEGMDLDVEQPMSQQGIDRLIARLRADFGPDFLITLAPVASALEDSSNLSGFSYTALQQTQGNDIDWYNTQFYSGFGSMADTSDYDRIVANGFAPAKVVAGQLTTPEGAGWIPTSSLNNTIVSLVSEYGQIGGVMGWEYFNSLPGGTAEPWEWAQIVTEILRPGLVPELKITEDDAARLTGAYEESVKAAAADNKSFVKRPSINYYAMVNA

SEQ ID NO: 11 - DNA, 1080 bp, Trichoderma reesei
  CDS (1)..(1080)
atgaaggcgtccgtctacttggcgtccctgctggcgacgctgtcgatggcggtgcccgtcaaggggctgcagctgcgggccgagccgacggacctgcctcgcctgattgtgtacttccagacgacgcacgacagcagcaaccggcccatctcgatgctgccgctcatcacggagaagggcatcgcgctgacgcacctcattgtgtgctcgttccacatcaaccaaggcggcgtggtgcacctcaacgacttcccgccggacgacccgcacttctacacgctgtggaacgagactatcacgatgaagcaggcgggcgtcaaggtcatgggcatggtgggcggcgcggcgccggggtcctttaacacgcagacgctcgactcgccggactcggccacgtttgagcactactacgggcagctgagggacgccattgtcaacttccagctcgagggcatggacctggacgtcgagcagccgatgagccagcagggcatcgaccggctgattgcgcggctgcgggcggatttcgggcccgacttcctcatcacgctggcgcccgtcgcgtcggcgctcgaggatagcagcaacctgtccggcttcagctacacggcgctgcagcagacgcagggcaacgacattgactggtacaacacgcagttctacagcggcttcggcagcatggcggacacgagcgactacgaccgcatcgtggccaacgggttcgcgcccgccaaggtggtggccggccagctgacggcgcccgagggcgcgggctggatcccgacgagcagcctcaacaacaccattgtctcgctcgtgagcgagtacggccagattggcggcgtcatgggctgggagtacttcaacagcctgcccggcggcaccgcggagccgtgggagtgggcgcagattgtgacgaagattctgaggccgggcttggtgccggagctgaagattacggaggacgatgcggcgaggctgacgagtgcgtatgaggagagcgtcaaggcggcggcggcggacaacaagagctttgtgaagaggcctagcattaactattatgctatggtgaatgcttaa

SEQ ID NO: 12 - protein, 359 aa, Trichoderma reesei
MKASVYLASLLATLSMAVPVKGLQLRAEPTDLPRLIVYFQTTHDSSNRPISMLPLITEKGIALTHLIVCSFHINQGGVVHLNDFPPDDPHFYTLWNETITMKQAGVKVMGMVGGAAPGSFNTQTLDSPDSATFEHYYGQLRDAIVNFQLEGMDLDVEQPMSQQGIDRLIARLRADFGPDFLITLAPVASALEDSSNLSGFSYTALQQTQGNDIDWYNTQFYSGFGSMADTSDYDRIVANGFAPAKVVAGQLTAPEGAGWIPTSSLNNTIVSLVSEYGQIGGVMGWEYFNSLPGGTAEPWEWAQIVTKILRPGLVPELKITEDDAARLTSAYEESVKAAAADNKSFVKRPSINYYAMVNA

SEQ ID NO: 13 - protein, 294 aa, Trichoderma reesei
AEPTDLPRLIVYFQTTHDSSNRPISMLPLITEKGIALTHLIVCSFHINQGGVVHLNDFPPDDPHFYTLWNETITMKQAGVKVMGMVGGAAPGSFNTQTLDSPDSATFEHYYGQLRDAIVNFQLEGMDLDVEQPMSQQGIDRLIARLRADFGPDFLITLAPVASALEDSSNLSGFSYTALQQTQGNDIDWYNTQFYSGFGSMADTSDYDRIVANGFAPAKVVAGQLTAPEGAGWIPTSSLNNTIVSLVSEYGQIGGVMGWEYFNSLPGGTAEPWEWAQIVTKILRPGLVPELKIT

SEQ ID NO: 14 - DNA, 22 bp, Artificial seqeunce
  forward PCR primer
gatgaaggcgtccgtctacttg

SEQ ID NO: 15 - DNA, 25 bp, artificial sequence
  reverse PCR primer
cgcccttatactctttgcctatttc

SEQ ID NO: 16 - protein, 7 aa, artificial sequence
  Endo T peptide sequence
AEPTDLP

SEQ ID NO: 17 - protein, 7 aa, artificial sequence
  Endo T peptide sequence
PGLVPEL

SEQ ID NO: 18 - protein, 14 aa, artificial sequence
  Endo T peptide sequence
TIDSPDSATFEHYY

SEQ ID NO: 19 - protein, 15 aa, artificial sequence
  Endo T peptide sequence
DIDVEQXXSQQGIDR

SEQ ID NO: 20 - protein, 5 aa, artificial sequence
  Endo T peptide sequence
AEPTD

SEQ ID NO: 21 - protein, 4 aa, artificial sequence
  Endo T peptide sequence
EIIR

SEQ ID NO: 22 - protein, 18 aa, artificial sequence
  Endo T peptide sequence
TIDSPDSATFEHYYXXXR

SEQ ID NO: 23 - protein, 26 aa, artificial sequence
  Endo T peptide sequence
DAIVNFXXXXXXIDVEQXXXQQGIDR

SEQ ID NO: 24 - protein, 9 aa, artificial sequence
  Endo T peptide sequence
DSPDSATXX

SEQ ID NO: 25 - protein, 26 aa, artificial sequence
  Endo T peptide sequence
VGGAAPGSFNTQTIDSPDSATFEHYY

SEQ ID NO: 26 - protein, 12 aa, artificial sequence
  Endo T peptide sequence
TIDSPDSATFEH

SEQ ID NO: 27 - protein, 18 aa, artificial sequence
  Endo T peptide sequence
TIDSPDSATFEHYYGQIR

SEQ ID NO: 28 - protein, 26 aa, artificial sequence
  Endo T peptide sequence
DAIVNFQLEGMDIDVEQPMSQQGIDR

SEQ ID NO: 29 - protein, 8 aa, artificial sequence
  Endo T peptide sequence
AEPTDLPR

SEQ ID NO: 30 - protein, 10 aa, artificial sequence
  Endo T peptide sequence
EILRPGLVPE

SEQ ID NO: 31 - protein, 4 aa, artificial sequence
  Endo T peptide sequence
DIPR

SEQ ID NO: 32 - protein, 7 aa, artificial sequence
  Endo T peptide sequence
HYYGQLR

SEQ ID NO: 33 - protein, 9 aa, artificial sequence
  Endo T peptide sequence
ILRPGLVPE

SEQ ID NO: 34 - protein, 16 aa, artificial sequence
  Endo T peptide sequence
GMDIDVEQPMSQQIDR

SEQ ID NO: 35 - protein, 8 aa, artificial sequence
  Endo T peptide sequence
XXDIDVEQ

SEQ ID NO: 36 - protein, 8 aa, artificial sequence
  Endo T peptide sequence
KQAGVKVM

SEQ ID NO: 37 - protein, 8 aa, artificial sequence
  Endo T peptide sequence
QQAGVQVM

SEQ ID NO: 38 - protein, 26 aa, artificial sequence
  Endo T peptide sequence
AEPTDLPRLIVYFQTTHDSSNRPISM

SEQ ID NO: 39 - protein, 7 aa, artificial sequence
  Endo T peptide sequence
QTTHDSS

SEQ ID NO: 40 - protein, 40 aa, artificial sequence
  Endo T peptide sequence
VGGAAPGSFNTQTLDSPDSATFEHYYGQLRDAIVNFQLEG

SEQ ID NO: 41 - protein, 18 aa, artificial sequence
  Endo T peptide sequence
LIVYFQTTHDSSNRPISM

SEQ ID NO: 42 - protein, 30 aa, artificial sequence
  Endo T peptide sequence
VGGAAPGSFNTQTLDSPDSATFEHYYGQLR

SEQ ID NO: 43 - protein, 10 aa, artificial sequence
  Endo T peptide sequence
IVANGFAPAK

SEQ ID NO: 44 - protein, 5 aa, artificial sequence
  ribonuclease peptide sequence
ANGFA

SEQ ID NO: 45 - protein, 17 aa, artificial sequence
  Endo T peptide sequence
GSLQDGQFVAAEPDGAK

SEQ ID NO: 46 - protein, 14 aa, artificial sequence
  Endo T peptide sequence
DIDVEQPMSQQIDR

SEQ ID NO: 47 - protein, 15 aa, artificial sequence
  Endo T peptide sequence
DIDVEQPMXXXXXDR

SEQ ID NO: 48 - protein, 11 aa, artificial sequence
  Endo T peptide sequence
YFQTTHDSSNR

SEQ ID NO: 49 - protein, 30 aa, artificial sequence
  Endo T peptide sequence
XXGAAPGSFNTQTIDSPDSATFEHYYXXXR

SEQ ID NO: 50 - protein, 9 aa, artificial sequence
  Endo T peptide sequence
IIRPGLVPE

SEQ ID NO: 51 - protein, 4 aa, artificial sequence
  Endo T peptide sequence
DDGE

SEQ ID NO: 52 - protein, 23 aa, artificial sequence
  Endo T peptide sequence
VGGAAPGSFNTQTLDSPDSATFE

SEQ ID NO: 53 - protein, 5 aa, artificial sequence
  Endo T peptide sequence
SDPSD

SEQ ID NO: 54 - protein, 4 aa, artificial sequence
  ribonuclease peptide sequence
VAAE

SEQ ID NO: 55 - protein, 17 aa, artificial sequence
  Endo T predicted signal sequence
MKASVYLASLLATLSMA

SEQ ID NO: 56 - DNA, 21 bp, artificial sequence
  sequence/pcr primer
ctgtaaagaggcttcaccccg

SEQ ID NO: 57 - DNA, 20 bp, artificial sequence
  sequence/pcr primer
ttcatgctctcatcacacag

SEQ ID NO: 58 - DNA, 35 bp, artificial sequence
  sequence/pcr primer
ggggatatcatatgaaggcgtccgtctacttggcg

SEQ ID NO: 59 - DNA, 38 bp, artificial sequence
  sequence/pcr primer
ggggatatctagataaagcattcaccatagcataatag

SEQ ID NO: 60 - DNA, 21 bp, artificial sequence
  sequence/pcr primer
acgcacctcattgtgtgctcg

SEQ ID NO: 61 - DNA, 21 bp, artificial sequence
  sequence/pcr primer
gtgggcggcgcggcgccgggg

SEQ ID NO: 62 - DNA, 21 bp, artificial sequence
  sequence/pcr primer
gaggatagcagcaacctgtcc

SEQ ID NO: 63 - DNA, 21 bp, artificial sequence
  sequence/pcr primer
ctcgtgagcgagtacggccag

SEQ ID NO: 64 - DNA, 18 bp, artificial sequence
  sequence/pcr primer
gaggagagcgtcaaggcg